(19) United States
(12) Patent Application Publication
(10) Pub. No.: US 2016/0195553 A1
(43) Pub. Date: Jul. 7, 2016

(54) INTELLIGENT TEST STRIP DETECTION DEVICE AND METHOD THEREOF

(71) Applicant: YONGLIN BIOTECH CORP., New Taipei City (TW)

(72) Inventors: Jiann-Hua Wang, New Taipei City (TW); Chen-Yu Lin, New Taipei City (TW)

(21) Appl. No.: 14/972,455

(22) Filed: Dec. 17, 2015

Related U.S. Application Data
(60) Provisional application No. 62/099,746, filed on Jan. 5, 2015.

(30) Foreign Application Priority Data
Apr. 16, 2015 (TW) 104112235

(51) Int. Cl. G01N 33/76; G01N 21/77; G06F 17/30; G01N 33/68

(52) U.S. Cl. CPC G01N 33/76; G01N 33/689; G01N 21/77; G06F 17/3056; G01N 2021/7759; G01N 2021/752

(57) ABSTRACT

Provided are an intelligent test strip detection device and method thereof. The intelligent test strip detection device includes a measuring unit, having a test strip end and a test strip reacting zone, wherein the test strip end tests a liquid with at least one kind of hormones, and the test strip reacting zone renders a result of the liquid under test; and a reading unit, having a slot, a signal processing circuit, a driver, a timing generator, a plurality of light sources, a plurality of photo sensors, and a connecting end, wherein the signal processing circuit is electrically connected to the driver, the timing generator, the plurality of light sources, and the plurality of photo sensors. Moreover, the measuring unit is inserted into the slot of the reading unit, and the connecting end of the reading unit is connected to a contact hole of a portable electronic device.

### CROSS REFERENCE TO RELATED APPLICATIONS

This application claims priority benefits of U.S. Provisional Patent Application No. 62/099,746 filed on Jan. 5, 2015 and Taiwanese Patent Application No. 104112235 filed on Apr. 16, 2015, which are hereby incorporated by reference in their entirety.

### BACKGROUND OF THE INVENTION

1. Field of the Invention

The present invention relates to a detecting device and a method, and relates particularly to an intelligent test strip detection device and a method thereof.

2. The Prior Arts

Following the changes of time and the rise of female self-awareness, there is an increasing trend of women in employment. However, in comparison with men, women in employment also face the pressure of reproduction besides facing the challenges at work. Therefore, working women need to put in extra effort, such that on the one hand they are able to work, and on the other hand they are able to think about starting a family.

In order to effectively grasp the critical period of conception in a busy working environment, newly married women usually make use of the ovulation agent 1 as shown in FIG. 1 to predict the ovulation date, thus increasing the chances of pregnancy. The indices that are mainly predicted by the conventional ovulation agent 1 include Follicle-stimulating hormone (FSH), luteinizing hormone (LH) and basal body temperature. The ovulation period can be grasped by predicting the indices mentioned above. However, since a lot of ovulation agents that are used in Taiwan are imported from Europe or the United States, therefore, the specifications of these ovulation agents comply with European or American specifications. The differences between the Europeans and Asians are therefore not considered. In terms of the ovulation period, there are differences in the values of FSH, LH and also values in the basal body temperature between European and American women as well as women from Asia. As such, if women from Asian countries directly use the ovulation agents that are imported from Europe or the United States, errors will always occur while using these ovulation agents.

In addition, the ovulation agents can mostly be used only once, and after measuring the physiological values each time, the user needs to make a record of and organize these physiological values. In other words, the use of ovulation agents can be quite inconvenient. In fact, following the advancement of technologies, the use of smartphones nowadays is very common, and almost everyone has one. Since the smartphone itself has a storage function, if the functions of the smartphone and the ovulation agents can be effectively combined, then (the) effective tracking of the date of ovulation can be significantly increased, thus providing the function of viewing personalized data.

As such, how to solve the problem of the differences observed in the measuring parameters of the conventional ovulation agents and how to provide personalized data on ovulation are the technical issues that are addressed by all sectors.

### SUMMARY OF THE INVENTION

In view of the drawbacks of the prior art, an objective of the present invention is to solve the problem of observing differences in the measuring parameters of the conventional ovulation agents, and to provide personalized data on ovulation.

In order to achieve the above-mentioned objectives, the present invention includes an intelligent test strip detection device, which includes a measuring unit, a test strip end and a test strip reaction zone, whereby the test strip end measures a test solution containing at least one physiological hormone. The test strip reaction zone is used for reacting with the test solution so as to display a detected result. The reading unit, which may include a slot, a signal processing circuit, a driver, a timing generator, a plurality of light sources, a plurality of light sensors and a connecting end. The signal processing circuit may be electrically connected to the driver, the timing generator, the plurality of light sources and the plurality of light sensors, whereby the measuring unit may be inserted into the slot of the reading unit; the connecting end of the reading unit may be connected to a connecting hole of the portable electronic device.

Preferably, the test solution may be urine from a human body, and may include a follicle-stimulating hormone (FSH) and a luteinizing hormone (LH).

Preferably, the measuring unit may also include a lid engaging portion, for covering the test strip end of the measuring unit.

Preferably, the timing generator may generate a plurality of clock control signals. The driver may drive the plurality of light sources in accordance with the plurality of time clock signals, to emit light to the test strip reaction zone. The plurality of light sensors may receive light from the test strip reaction zone, in accordance with the plurality of clock control signals, so as to generate a detecting signal. The signal processing circuit may process the detecting signal.

Preferably, the signal processing circuit, the driver and the timing generator may be integrated into an integrated circuit, the plurality of light sources, and the plurality of light sensors may be installed on a printed circuit board.

Preferably, the portable electronic device may include a display unit, for displaying data on the ovulation date; the portable electronic device may also include a storing unit, for storing the data of the ovulation date; and an application module, for enabling the reading unit to read the detected result of at least one physiological hormone.

Preferably, a ratio of the FSH to LH may be calculated automatically, in accordance with a plurality of detected results.

Preferably, the connecting end of the reading unit may be a Micro-USB connecting end.

The present invention also provides a detection method of an intelligent test strip, which includes the following steps: inserting a measuring unit to a test solution containing at least one physiological hormone, for reacting with the at least one physiological hormone, so as to display the detected result; inserting the measuring unit into a reading unit; connecting the connecting end of the reading unit to the connecting hole of the portable electronic device; and enabling the reading unit to read the detected result of the at least one physiological hormone by the portable electronic device, by means of the application module, whereby the reading unit may include a slot, a signal processing circuit, a driver, a timing generator, a plurality of light sources, a plurality of light sensors and a connecting end.

Preferably, the application module may be remotely connected to a cloud database through the Internet.

The present invention makes use of the physiological values such as the values of FSH and LH to predict the ovulation date of women, and has greater accuracy in comparison with the prior art. Moreover, the portable electronic device of the present invention is used to manage the personalized physiological data, and can be more convenience in use. As such, the intelligent test strip detection device and method thereof have greater accuracy in predicting the ovulation date of women, and have the advantages of providing a professional and personalized service, and therefore enabling the drawbacks of the prior art to be improved.

### BRIEF DESCRIPTION OF THE DRAWINGS

The present invention can be understood in more detail by reading the subsequent detailed description in conjunction with the examples and references made to the accompanying drawings, wherein:

FIG. 1 is a schematic diagram showing the conventional ovulation agent;

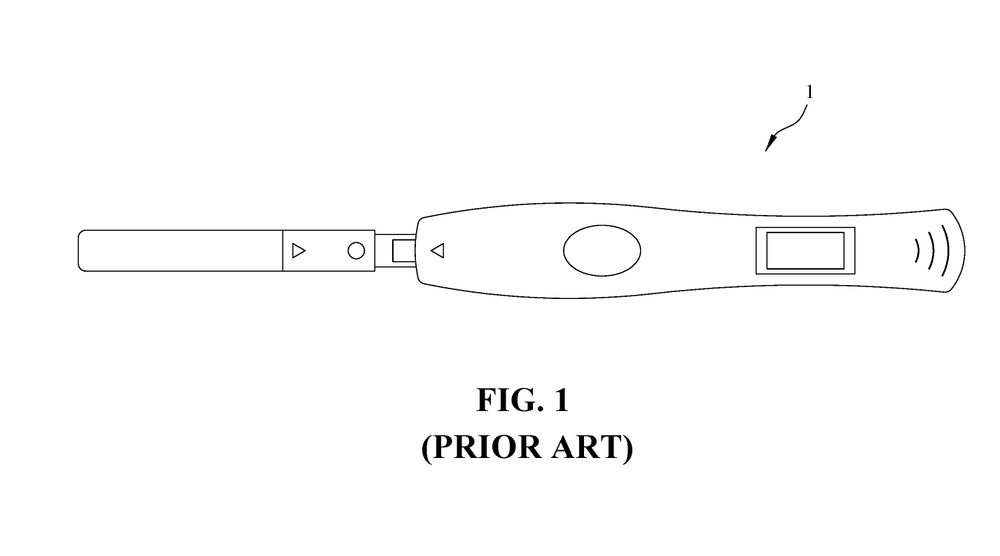

FIG. 2 is a schematic diagram showing the intelligent test strip detection device in accordance with an embodiment of the present invention;

FIG. 3 is a system block diagram of the reading unit in accordance with an embodiment of the present invention;

FIG. 4 is a schematic diagram showing another intelligent test strip detection device in accordance with an embodiment of the present invention; and

FIG. 5 is a flow chart showing the method of the intelligent test strip detection device of the present invention.

### DETAILED DESCRIPTION OF THE PREFERRED EMBODIMENT

The present invention may be embodied in various forms and the details of the preferred embodiments of the present invention will be described in the subsequent content with reference to the accompanying drawings. The drawings (not to scale) depict only the preferred embodiments of the invention and shall not be considered as limitations to the scope of the present invention. Modifications of the shape of the present invention shall be considered within the spirit of the present invention.

It should be noted that the structures, proportions, sizes and the like of the drawings of the current specification are only for depicting the disclosures of the present invention, to enable easy reading and understanding by persons skilled in the art, and these are not meant to be conditions that limit the present invention. As such, the drawings are not technologically meaningful. Any modifications of the structure, proportion relationships or any adjustments of the sizes should fall within the scope that is covered by the technical content of the present invention, without affecting the effects produced by the present invention and without affecting the goals of the present invention.

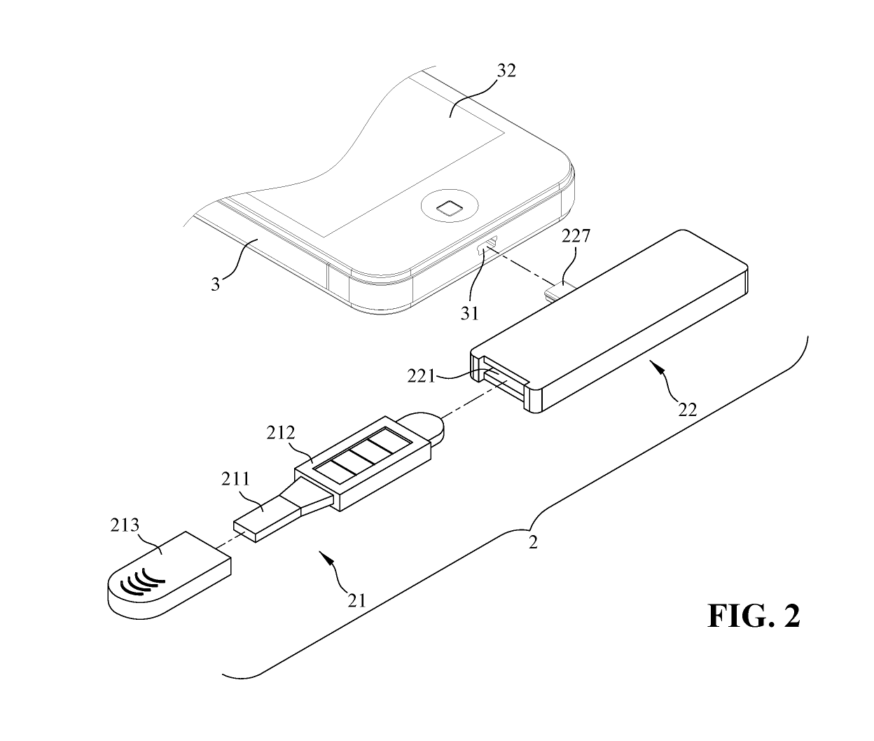

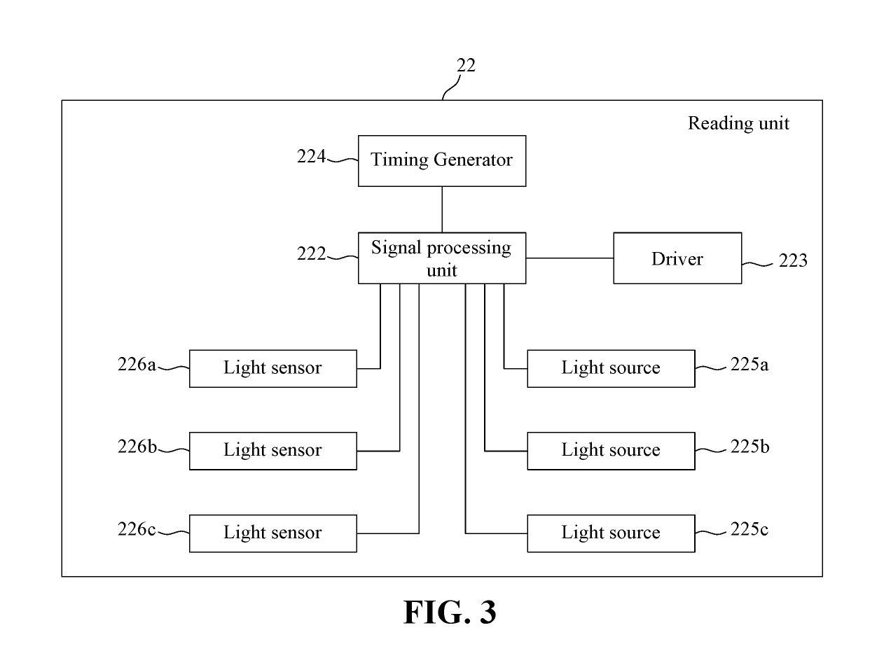

Please refer to FIG. 2 and FIG. 3 together. FIG. 2 is a schematic diagram showing the intelligent test strip detection device in accordance with an embodiment of the present invention, and FIG. 3 is a system block diagram of the reading unit in accordance to an embodiment of the present invention. In accordance with a preferred embodiment of the present invention, the intelligent test strip detection device 2 of the present invention includes: a measuring unit 21, which may include a test strip end 211 and a test strip reaction zone 212; whereby the test strip end 211 may measure a test solution containing at least one physiological hormone (not shown in the drawings). In addition, the test strip reaction zone 212 may be used for reacting with a test solution, so as to display the detected results. The intelligent test strip detection device of the present invention may also include a reading unit 22. The reading unit 22 may include a slot 221, a signal processing circuit 222, a driver 223, a timing generator 224, a plurality of light sources 225a-225c, a plurality of light sensors 226a-226c and a connecting end 227. The signal processing circuit 222 may be electrically connected to the driver 223, the timing generator 224, the plurality of light sources 2251-225c and the plurality of light sensors 226a-226c. Moreover, the measuring unit 21 may be inserted into the slot 221 of the reading unit 22. The connecting end 227 of the reading unit 22 may be connected to the connecting hole 31 of the portable electronic device 3.

In a preferred embodiment of the present invention, the test solution may be urine from a human body, and may contain Follicle-stimulating hormone (FSH) and luteinizing hormone (LH).

In a preferred embodiment of the present invention, the measuring unit 21 may include a lid engaging portion 213, so as to cover the test strip end 211 of the measuring unit 21.

In a preferred embodiment of the present invention, the timing generator 224 may produce a plurality of watch control signals. In accordance with the plurality of clock control signals, the driver 223 may drive the plurality of light sources 225a-225c, so as to emit light to the test strip reaction zone 212. In accordance with the plurality of clock control signals, the plurality of light sensors 226a-226c may receive light from the test strip reaction zone 212, so as to produce a detecting signal. Also, the signal processing circuit 222 may process the detecting signal.

In a preferred embodiment of the present invention, the signal processing circuit 222, the driver 223 as well as the timing generator 224 may be integrated into an integrated circuit (not shown in the drawings). In addition, the plurality of light sources 225a-225c and the plurality of light sensors 226a-226c may be installed on a printed circuit board (not shown in the drawings).

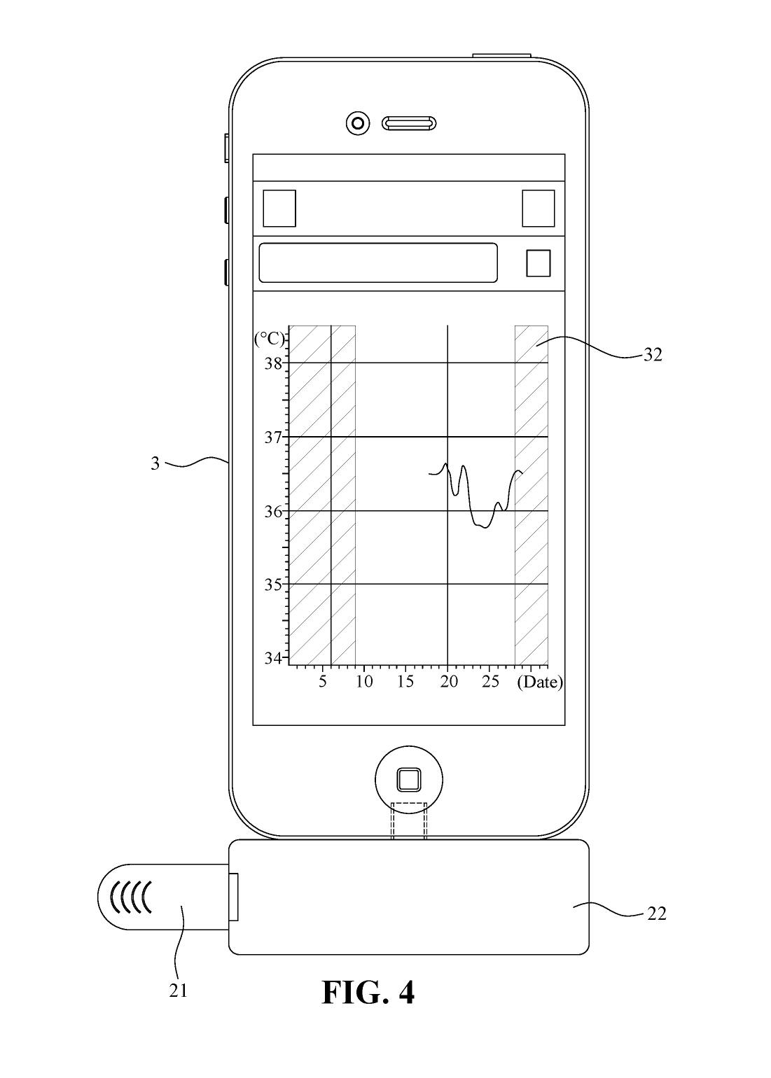

In a preferred embodiment of the present invention, the portable electronic device 3 may include a display unit 32. The display unit displays data about the ovulation date. As shown in FIG. 4, the storing unit (not shown in the drawings) stores data about the ovulation date. The portable electronic device also includes an application module (not shown in the drawings), which is for the reading unit 22 to read the detection result of the at least one physiological hormone.

In a preferred embodiment of the present invention, the ratio of FSH and LH may be automatically calculated, in accordance with the plurality of detected results.

In a preferred embodiment of the present invention, the connecting end 227 of the reading unit 22 may be a Micro-USB connecting end.

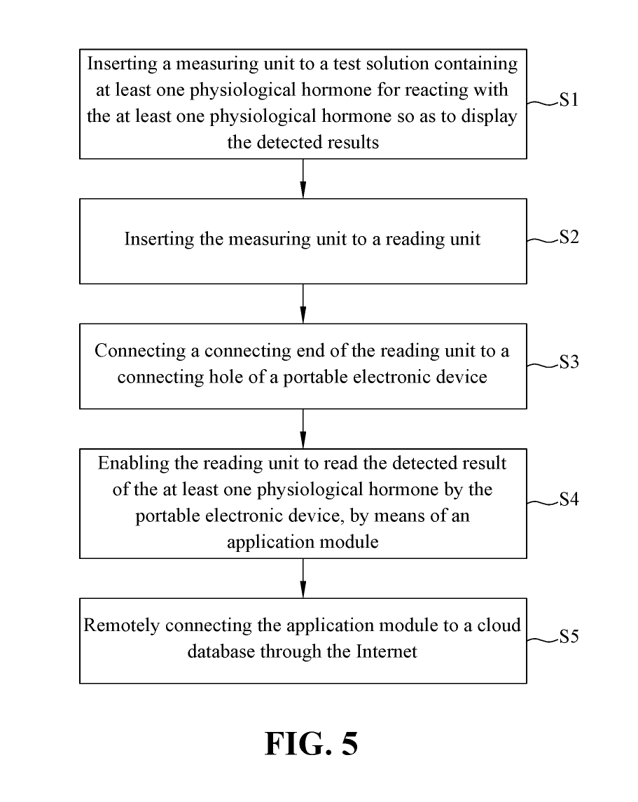

In addition, the present invention also provides a detection method of an intelligent test strip. Referring to FIG. 5, the method of the present invention may include the following steps: inserting a measuring unit to a test solution containing at least one physiological hormone, so as to react with the at least one physiological hormone, and so as to display the detected results (Step S1); inserting the measuring unit to the interior of a reading unit (Step S2); connecting a connecting end of the reading unit to a connecting hole of the portable electronic device (Step S3); and enabling the reading unit to read the detected results of the at least one physiological hormone, by means of the portable electronic device and an application module (Step S4). The reading unit may include a slot, a signal processing circuit, a driver, a timing generator, a plurality of light sources, a plurality of light sensors as well as a connecting end.

The method of the present invention may further include an application module that may be remotely connected to a cloud database, by means of the internet (Step S5).

The intelligent test strip detection device and method thereof provided by the present invention make use of physiological values of FSH and LH to predict the ovulation date of women, and have greater accuracy in comparison with those of the prior art. Moreover, the use of the portable electronic device enables the management of the physiological data, enabling the user to have personalized management of the data. The present invention not only solves the problem of the inadequacies of the measuring parameters of the conventional ovulation agents, the present invention has also solved the problems such as the ineffective personalization and customization of ovulation data of women.

Although the present invention has been described with reference to the preferred embodiments, it will be understood that the invention is not limited to the details described thereof. Various substitutions and modifications have been suggested in the foregoing description, and others will occur to those of ordinary skill in the art. Therefore, all such substitutions and modifications are intended to be embraced within the scope of the invention as defined in the appended claims.

What is claimed is:

1. An intelligent test strip detection device, comprising:
a measuring unit, including a test strip end and a test strip reaction zone, wherein the test strip end measures a test solution containing at least one physiological hormone, and the test strip reaction zone is used for reacting with the test solution so as to display a detected result; and
a reading unit, including a slot, a signal processing circuit, a driver, a timing generator, a plurality of light sources, a plurality of light sensors and a connecting end, wherein the signal processing circuit is electrically connected to the driver, the timing generator, the plurality of light sources and the plurality of light sensors;
wherein the measuring unit is inserted into the slot of the reading unit, the connecting end of the reading unit is connected to a connecting hole of the portable electronic device.

2. The intelligent test strip detection device of claim 1, wherein the test solution is urine from a human body, and comprising a follicle-stimulating hormone (FSH) and a luteinizing hormone (LH).

3. The intelligent test strip detection device of claim 1, wherein the measuring unit further comprises a lid engaging portion, for covering the test strip end of the measuring unit.

4. The intelligent test strip detection device of claim 1, wherein the timing generator generates a plurality of clock control signal; the driver drives the plurality of light sources in accordance with the plurality of time clock signals, to emit light to the test strip reaction zone; the plurality of light sensors receive light from the test strip reaction zone, in accordance with the plurality of clock control signals, so as to generate a detecting signal; and the signal processing circuit processes the detecting signal.

5. The intelligent test strip detection device of claim 1, wherein the signal processing circuit, the driver and the timing generator are integrated into an integrated circuit, and the plurality of light sources, and the plurality of light sensors are installed on a printed circuit board.

6. The intelligent test strip detection device of claim 1, wherein the portable electronic device comprises a display unit, for displaying data on the ovulation date; a storing unit, for storing the data of the ovulation date; and an application module, enabling the reading unit to read a detection result of at least one physiological hormone.

7. The intelligent test strip detection device of claim 2, wherein a ratio of a FSH to a LH is calculated automatically, in accordance with a plurality of detected results.

8. The intelligent test strip detection device of claim 1, wherein the connecting end of the reading unit is a Micro-USB connecting end.

9. A detection method of an intelligent test strip, comprising the steps of:
inserting a measuring unit to a test solution comprising at least one physiological hormone, for reacting with the at least one physiological hormone so as to display the detected result;
inserting the measuring unit into a reading unit;
connecting a connecting end of the reading unit to a connecting hole of a portable electronic device; and
enabling the reading unit to read the detected result of the at least one physiological hormone by the portable electronic device, by means of an application module;
wherein the reading unit comprises a slot, a signal processing circuit, a driver, a timing generator, a plurality of light sources, a plurality of light sensors and a connecting end.

10. The detection method of the intelligent test strip of claim 9, wherein the application module remotely connects to a cloud database through the Internet.
